(19) United States
(12) Patent Application Publication
(10) Pub. No.: US 2022/0333111 A1
(43) Pub. Date: Oct. 20, 2022

(54) ANTI-MIRNAS FOR THE TREATMENT OF LEIOMYOMA

(71) Applicant: INTERNATIONAL CENTRE FOR GENETIC ENGINEERING AND BIOTECHNOLOGY - ICGEB, Trieste (IT)

(72) Inventors: Serena ZACCHIGNA, Trieste (IT); Nadja RING, Trieste (IT)

(21) Appl. No.: 17/763,303

(22) PCT Filed: Sep. 23, 2020
(86) PCT No.: PCT/EP2020/076498

§ 371 Date: Mar. 24, 2022

(30) Foreign Application Priority Data
Sep. 25, 2019 (IT) 102019000017234

(51) Int. Cl. C12N 15/113; A61K 41/00; A61P 15/00

(52) U.S. Cl. CPC C12N 15/113; A61K 41/0047; A61P 15/00; C12N 2310/141; C12N 2310/3231

(57) ABSTRACT

The present invention relates to miRNA inhibitors for use for the treatment of leiomyoma. In particular, it refers to an inhibitor of one or more miRNAs selected from the group consisting of miR-148a-3p, miR-199a-5p and miR-33b-3p for use in the treatment of uterine leiomyoma. Said inhibitor is preferably an LNA-based oligonucleotide. Pharmaceutical compositions comprising said inhibitor for use for the treatment of uterine leiomyoma are also within the invention.

### FIELD OF INVENTION

The present invention refers to the pharmaceutical and biotechnology fields.

In particular, the present invention refers to anti-miRNAs for use in the treatment of leiomyoma.

### BACKGROUND OF THE INVENTION

Uterine leiomyomas, commonly called fibroids, affect 20-50% of women in their reproductive age, representing the most frequent form of gynecological benign tumor (Purohit P, Vigneswaran K: Fibroids and Infertility, Curr Obstet Gynecol Rep 2016, 5:81-88).

Although the consequences of fibroids on women reproductive function and fertility are not fully established, it is generally accepted that their presence significantly interferes with the ability to successfully start a pregnancy (Pritts EA, Parker WH, Olive DL: Fibroids and infertility: an updated systematic review of the evidence, Fertil Steril 2009, 91:1215-1223). Although they are benign tumors, and can even be asymptomatic, in many cases they are responsible for important dysfunctions, such as bleeding, pelvic pressure and, as mentioned above, impaired fertility, thus requiring therapeutic intervention.

Currently, the therapy of fibroids is based on the use of drugs that interfere with the hormonal stimulation underlying their growth and sometimes manage to control the symptoms, and on surgical removal.

Hysterectomy, the surgical removal of the uterus, is the only definitive solution to the problem. However, in many cases this solution is not feasible, for example if the woman is planning a pregnancy or simply for psychological reasons. In these cases, local removal of the neoplastic mass has been the primary treatment choice over the last 100 years, initially by laparotomy and, more recently, in a less invasive manner, by laparoscopy or hysteroscopy (El-Balat A, DeWilde RL, Schmeil I, Tahmasbi-Rad M, Bogdanyova S, Fathi A, Becker S: Modern Myoma Treatment in the Last 20 Years: A Review of the Literature, Biomed Res Int 2018, 2018:4593875).

Any surgical intervention is always associated with a limited but real risk of complications, such as bleeding (which in turn often requires blood transfusion with a consequent risk of transmission of infectious agents), bladder, bowel or ureteral lesions, adhesion formation, complications related to anesthesia and hospitalization.

Current drug therapies, including oral contraceptives, medicalized intrauterine devices, GnRH agonists and selective progesterone modulators (SPRMs) are mainly used to control symptoms (bleeding and dysmenorrhea), without interfering with the mass size of the leiomyoma (Senol T, Kahramanoglu I, Dogan Y, Baktiroglu M, Karateke A, Suer N: Levonorgestrel-releasing intrauterine device use as an alternative to surgical therapy for uterine leiomyoma, Clin Exp Obstet Gynecol 2015, 42:224-227; Singh SS, Belland L: Contemporary management of uterine fibroids: focus on emerging medical treatments, Curr Med Res Opin 2015, 31:1-12).

Even the latest generation drugs, such as ulipristil acetate, are able to control bleeding in the absence of significant side effects, but are not able to reduce the size of the lesions (Donnez J, Tatarchuk TF, Bouchard P, Puscasiu L, Zakharenko NF, Ivanova T, Ugocsai G, Mara M, Jilla MP, Bestel E, Terrill P, Osterloh I, Loumaye E, Group PIS: Ulipristal acetate versus placebo for fibroid treatment before surgery, N Engl J Med 2012, 366:409-420; Donnez J, Tomaszewski J, Vazquez F, Bouchard P, Lemieszczuk B, Baro F, Nouri K, Selvaggi L, Sodowski K, Bestel E, Terrill P, Osterloh I, Loumaye E, Group PIS: Ulipristal acetate versus leuprolide acetate for uterine fibroids, N Engl J Med 2012, 366:421-432).

There are currently no drug therapies that can reduce the size of leiomyomas.

Therefore, the development of new conservative therapies, capable of stopping or regressing the growth of leiomyomas, is necessary.

In particular, the development of a drug capable of reducing the size of leiomyomas without interfering with the ovulatory cycles and fertility of the woman is desirable.

From a morphological point of view, leiomyomas are benign hyperplastic lesions of the smooth muscle cells of the uterus. Despite their frequency, the cause of the aberrant proliferation of smooth muscle cells remains unknown.

Several theories have been proposed in an attempt to explain the etiology of these lesions, which suggest the involvement of estrogens and progesterone, but also of several other growth factors, cytokines, chemokines, genes and microRNA (miRNA).

In particular, miRNAs are small RNA molecules, approximately 21 nucleotides long, that regulate the expression of different genes, binding to the untranslated region at the 3′ end of the corresponding mRNAs.

MiRNAs can be inhibited using specific LNA (locked-nucleic acid)-based oligonucleotides or antagomiRs, i.e. oligonucleotides with a sequence complementary to that of miRNA. These oligonucleotides typically contain chemical modifications that increase their affinity to the target mRNA molecules and trap them in an inactive configuration or promote degradation.

In general, miRNA-based biological drugs, or inhibitors thereof, are useful tools to interfere with the processes underlying several diseases.

### SUMMARY OF THE INVENTION

It has now been found that the inhibition of any one of the following miRNAs is able to block the proliferation of primary leiomyoma cells: miR-148a-3p, miR-199a-5p, miR-33b-3p.

Therefore, an object of the invention is an inhibitor of one or more miRNAs selected from the group consisting of miR-148a-3p, miR-199a-5p and miR-33b-3p for use in the treatment of uterine leiomyoma.

Said inhibitor is preferably chosen between a locked-nucleic acid (LNA)-based oligonucleotide and an anti-miR. Preferably, it is an LNA-based oligonucleotide.

A pharmaceutical composition comprising said inhibitor and at least one pharmaceutically acceptable vehicle and/or excipient for use in the treatment of uterine leiomyoma is also within the scope of the present invention.

### DESCRIPTION OF THE INVENTION

### Definitions

Within the context of the present invention, the term “microRNA” or “miRNA” means a short ribonucleic acid (RNA) present in eukaryotic cells.

Within the context of the present invention, the term “inhibitor” means an agent capable of decreasing or blocking the activity of one or more target miRNAs. In particular, it means an agent capable of sufficiently decreasing the activity of one or more target miRNAs. In a particular embodiment, an inhibitor according to the present invention is an oligonucleotide comprising at least 5 nucleotides capable of binding to a target miRNA by complementary base pairing, thereby decreasing or blocking the function of said miRNA. For “complementary base pairing” it is intended that some or all of the bases of said oligonucleotide pair with some or all of the bases of said miRNA target. In particular, said inhibitor can be able to pair at least 40% of its bases with the bases of the target miRNA; for example said inhibitor is able to pair at least 40%, 50%, 60%, 70%, 80%, 90% or 100% of its bases with the bases of the target miRNA. The term “base” means a nucleobase, i.e. the nitrogen-containing portion of nucleosides, which in turn are components of nucleotides.

Within the context of the present invention, the term “anti-miR” or “antagomir” means an oligonucleotide molecule that prevents the binding of other molecules to one or more specific miRNAs. Generally, it is a small synthetic RNA at least partially complementary to the target miRNA. In particular, it is sufficiently complementary to bind and block said target miRNA.

Within the context of the present invention, the term “locked nucleic acid” or “LNA” means a nucleotide with the ribose ring blocked in an N-type conformation by a 2′-O, 4′-C bridge. LNAs are described for example in WO 99/14226, WO 00/56746, WO 00/56748, WO 01/25248, WO 02/28875, WO 03/006475 and WO 03/095467 and the references mentioned therein.

Within the context of the present invention, the term “LNA-based oligonucleotide” means an oligonucleotide comprising at least one locked nucleic acid (LNA), as defined above. This oligonucleotide is generally a small synthetic RNA at least partially complementary to the target miRNA, in particular sufficiently complementary to bind and block the target miRNA, including at least one locked nucleic acid.

Within the context of the present invention, the term “uterine leiomyoma” means a benign tumour of the uterus. “Uterine fibroma” is a synonym.

### FIGURES

FIG. 1: Proliferation of human smooth muscle cells, untreated or after transfection with a control miRNA or with a LNA-based oligonucleotide capable of inhibiting miR-148a-3p, miR-199a-5p and miR-33b-3p (*p≤0.05; ***p≤0.001).

FIG. 2: Proliferation of human fibroblasts, untreated or after transfection with a control miRNA or with a LNA-based oligonucleotide capable of inhibiting miR-148a-3p, miR-199a-5p and miR-33b-3p (ns, not significant).

FIG. 3: Proliferation of human smooth muscle cells, untreated or after administration under gymnosis conditions (in the absence of a transfection agent) with a control miRNA or with a LNA-based oligonucleotide capable of inhibiting miR-148a-3p, miR-199a-5p and miR-33b-3p (* p≤0.05).

### DETAILED DESCRIPTION OF THE INVENTION

The sequences of the miRNAs miR-148a-3p, miR-199a-5p, and miR-33b-3p are known to the experts in the field, for example they can be found in the miRbase database (www.mirbase.org, release 22).

According to this invention, at least one miRNA chosen between miR-148a-3p, miR-199a-5p, and miR-33b-3p is inhibited.

The inhibition of these miRNAs is particularly advantageous because it shows cell specificity, indeed it induces a decrease of the proliferation of uterine smooth muscle cells without affecting the proliferation of fibroblasts, thus reducing possible side effects.

Even more preferably, miR-148a-3p is inhibited. Indeed, inhibition of this miRNA was found particularly effective in reducing the proliferation of uterine muscle cells.

According to the present invention, an inhibitor of one or more of said miRNAs is used.

Any inhibitor capable of decreasing or blocking the activity of one or more of the above listed miRNAs may be used according to this invention for the treatment of uterine leiomyoma.

In a particular embodiment, said inhibitor is an oligonucleotide comprising at least 5 nucleotides and able of binding to a target miRNA by complementary base pairing. For example, said oligonucleotide can comprise between 5 and 27 nucleotides, preferably between 10 and 21 nucleotides.

Said oligonucleotide is able to pair some or all of its bases with some or all of the bases of said miRNA(s) target. In particular, said inhibitor can be able to pair at least 40% of its bases with the bases of the target miRNA(s); for example it pairs at least 40%, 50%, 60%, 70%, 80%, 90% or 100% of its bases with the bases of the target miRNA(s).

In a preferred embodiment, the inhibitor is chosen between a locked-nucleic acid (LNA)-based oligonucleotide and an anti-miR.

In an embodiment, the inhibitor is an anti-miR wherein said anti-miR is an RNA oligonucleotide at least partially complementary to the target miRNA. In particular, it is sufficiently complementary to bind and at least partially block said target miRNA. Preferably, said anti-miR comprises between 5 and 27 nucleotides and it has at least 40% of the bases complementary with the bases of the target miRNA.

In a preferred embodiment, said inhibitor is an LNA-based oligonucleotide, as defined above. Preferably, it comprises between 5 and 27 nucleotides and it comprises at least one locked nucleic acid. It can also comprise more than one locked nucleic acid. Said LNA-based oligonucleotide is at least partially complementary to the miRNA target, in particular it can be at least 40%, 50%, 60%, 70%, 80%, 90% or 100% complementary to the miRNA target.

LNA-based oligonucleotides are inhibitors of common use in the field and commercially available. For example, they are available from the following companies: QIAgen, Affymetrix, Perkin Elmer.

In a particular embodiment of the invention, said LNA-based oligonucleotide is an oligonucleotide, in particular an antisense oligonucleotide, with perfect sequence complementary to the miRNA target. Said LNA-based oligonucleotide can be 100% complementary to the miRNA target. For example, it can be an LNA-based oligonucleotide with perfect sequence complementary to the miRNA target of the type miRCURY LNA miRNA Power Inhibitor, commercially available from QIAgen.

The expert in the field is able to find an inhibitor suitable for use according to this invention according to the general knowledge in the field.

Indeed, it is well known in the field how to identify an agent able to inhibit the activity of a known miRNA. In particular, anti-miR and LNA-based oligonucleotides are commonly used to inhibit the activity of target miRNAs. A skilled person in the field can easily design or purchase anti-miR or LNA-based oligonucleotides able to inhibit the activity of at least one miRNA chosen between miR-148a-3p, miR-199a-5p, and miR-33b-3p.

The inhibitor for use as described in the present invention may be administered to a subject in need thereof in any form.

In particular, it may be administered by conventional methods of administration of small RNAs.

In an embodiment, it is administered without the use of transfection agents.

In an alternative embodiment, it is administered in a pharmaceutical composition.

Said pharmaceutical composition may be in the form of a preparation for parenteral or intrauterine administration, but other forms are equally suitable for carrying out the present invention.

The expert in the field will decide the effective timing of administration, depending on the patient's condition, the level of severity of the pathology, the patient's response and any other clinical parameters included in the general knowledge in the field.

The pharmaceutical composition contains at least one inhibitor according to this invention together with a pharmaceutically acceptable vehicle and/or excipient. Said excipient may be a particularly useful formulation adjuvant, e.g. a solubilizing agent, dispersing agent, suspending agent or emulsifier.

According to the present invention, the inhibitor can be administered together with lipid molecules, such as cationic lipids that can facilitate its transport, according to the state of the art. A further method of administering such an inhibitor is by means of a suitable vector, known for the administration of RNA or DNA. A preferred vector is the adeno-associated vector (AAV), a viral vector well known for in vivo administration of DNA (Mingozzi F, High KA: Therapeutic in vivo gene transfer for genetic disease using AAV: progress and challenges. Nature reviews genetics. May 2011;12(5):341).

Injection is a preferred route of administration. Injection is preferably systemic. Intra-uterine administration is also advantageous. An expert in the field may decide to administer the inhibitor by any conventional route.

For general knowledge in the field, reference can be made to Remington's Pharmaceutical Sciences, latest edition.

A further possible method of administration comprises the use of gene transfer technology with non-viral ultrasound (sonoporation), which can be used to locally transfect uterine cells with an inhibitor according to the present invention. Such technology is well known, reference can be made for example to the work Acoustic Cavitation-Mediated Delivery of Small Interfering Ribonucleic Acids with Phase-Shift Nano-Emulsions; Ultrasound Med Biol. Aug. 2015;41(8):2191-201.

A further mode of administration is through the use of a medicated intrauterine device for the release of the inhibitor. For example, said intrauterine device can be medicated with at least one locked-nucleic acid (LNA)-based oligonucleotide or with at least one anti-miR, as above defined, able to inhibit said one or more miRNAs. Such medicated intrauterine device for the use herein disclosed is within the scope of the invention.

All these methods and formulations are conventional and well known in the field and require no further explanation.

Gene therapy is another form of delivery, wherein nucleic acids are delivered into cells of a subject in need thereof. According to the present invention, gene therapy can be used to introduce an oligonucleotide inhibitor as above defined into a subject's cells, for example into uterine cells, for the use according to the present invention.

The following examples further illustrate the invention.

### EXAMPLE 1

### Selection of miRNA candidates

A screening was performed using a library of 2000 miRNAs to identify miRNAs capable of modulating the proliferation of a line of smooth muscle cells. In particular, the screening aimed at identifying miRNAs capable of stimulating the proliferation of smooth muscle cells.

The results of the screening are shown in Table 1 below.

Of the miRNAs considered in the screening, 1913 did not interfere with cell viability and 37 increased proliferation by more than 2.5 times.

By cross-referencing these data with those available in published databases of gene expression profiles of leiomyoma and normal myometrium samples (Chuang TD, Khorram O: Expression Profiling of IncRNAs, miRNAs, and mRNAs and Their Differential Expression in Leiomyoma Using Next-Generation RNA Sequencing, Reprod Sci 2018, 25:246-255), 14 miRNAs were selected (Table 1).

TABLE 1

| | | | Expression in | Average | |
|---|---|---|---|---|---|
| | | | Leiomyoma/ | number of | |
| | SMC fold | Ranking by | healthy | reads in | Seed |
| v21.0-miRNA | proliferation | proliferation | Myometrium | leiomyoma | sequence |
| hsa-miR-152-3p | 9.3 | 1 | 1.1 | 4347.7 | CAGUGC |
| hsa-miR-148a-3p | 7.3 | 3 | 1.1 | 122009.7 | CAGUGC |
| hsa-miR-199b-5p | 7.0 | 4 | 1.1 | 4794.7 | CCAGUG |
| hsa-miR-148b-3p | 5.5 | 6 | 1.2 | 4237.3 | CAGUGC |
| hsa-miR-199a-5p | 5.2 | 8 | 1.4 | 14935.0 | CCAGUG |
| hsa-miR-106b-5p | 4.1 | 11 | 1.6 | 63.0 | AAAGUG |
| hsa-miR-106a-5p | 3.5 | 17 | 1.9 | 10.7 | AAAGUG |
| hsa-miR-372-3p | 3.5 | 18 | 2.5 | 1.7 | AAGUGC |
| hsa-miR-6716-3p | 3.4 | 21 | 0.6 | 1.3 | CCGAAC |
| hsa-miR-20a-5p | 3.3 | 22 | 1.3 | 556.0 | AAAGUG |
| hsa-miR-17-5p | 3.3 | 24 | 1.4 | 252.7 | AAAGUG |
| hsa-miR-873-3p | 3.0 | 29 | 0.8 | 27.7 | GAGACU |
| hsa-miR-93-5p | 2.7 | 34 | 1.5 | 379.3 | AAAGUG |
| hsa-miR-33b-3p | 2.6 | 36 | NA | 0.7 | AGUGCC |

Of the 14 miRNAs selected, only seven “seed sequences” are represented, suggesting that their pro-proliferative action could be mediated by common molecular mechanisms. Furthermore, two miRNAs, miR-20a-5p and miR-17-5p, belong to the same cluster, located on chromosome 13, and therefore are expected to be co-regulated in their expression.

From the same selection the following miRNAs were considered for further validation: miR-148a-3p, miR-199a-5p, miR-20a-5p, miR-17-5p, and miR-33b-3p. MiR-148a-3p and miR-199a-5p were chosen because they were placed in second and fifth position in screening, are the most expressed in leiomyomas, and are also over-expressed in leiomyomas compared to healthy myometrium. Two additional miRNAs, miR-20a-5p and miR-17-5p are known to stimulate proliferation of other cell types and were chosen as potential positive controls. Finally, miR-33b-3p was found to be relatively poorly expressed in leiomyomas but completely absent in healthy myometrium and therefore included among the potentially interesting candidates.

### EXAMPLE 2

### In vitro validation

Based on the results reported in example 1, we tested the ability of five candidate LNA-based oligonucleotides (miRCURY LNA miRNA Power Inhibitor, Qiagen) to block the proliferation of primary leiomyoma cells, obtained through an existing collaboration with the Burlo Garofolo maternal and child hospital in Trieste.

The LNA-based oligonucleotides were tested using a standard transfection protocol with cationic lipids, or under gymnosis conditions (without the use of transfection agents) to better mimic a possible in vivo application (for the procedures used see for example Single-Dose Intracardiac Injection of Pro-Regenerative MicroRNAs Improves Cardiac Function After Myocardial Infarction. Lesizza P, Prosdocimo G, Martinelli V, Sinagra G, Zacchigna S, Giacca M. Circ Res. Apr. 14, 2017;120(8):1298-1304; Efficient gene silencing by delivery of locked nucleic acid antisense oligonucleotides, unassisted by transfection reagents. Stein CA, Hansen JB, Lai J, Wu S, Voskresenskiy A, Høg A, Worm J, Hedtjärn M, Souleimanian N, Miller P, Soifer HS, Castanotto D, Benimetskaya L, Ørum H, Koch T. Nucleic Acids Res. Jan. 2010;38(1):e3). Proliferation was analyzed through the incorporation of EdU and subsequent quantification by fluorescence microscopy (ImageXpress Micro, Molecular Devices) for the visualization of smooth muscle cells marked with an antibody recognising an isoform of actin which is specific for smooth muscle. The nuclei of all cells were stained with DAPI.

The inhibitors (3 candidates plus 2 positive controls) were tested in both leiomyoma cells and primary fibroblasts to verify their specificity of action, using 17-5p LNA and 20a-5p LNA as positive controls.

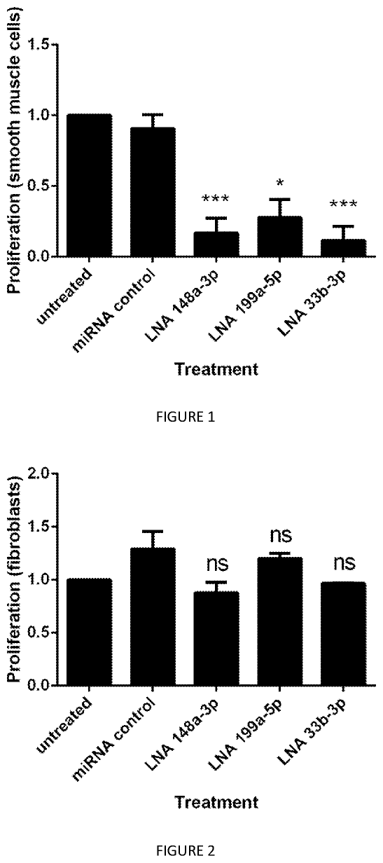

These experiments enabled the selection of three LNA-based oligonucleotides which are extremely potent in inhibiting the proliferation of leiomyoma cells and are not active in fibroblasts: LNA 148a-3p, LNA 199a-5p and LNA 33b-3p (FIGS. 1 and 2).

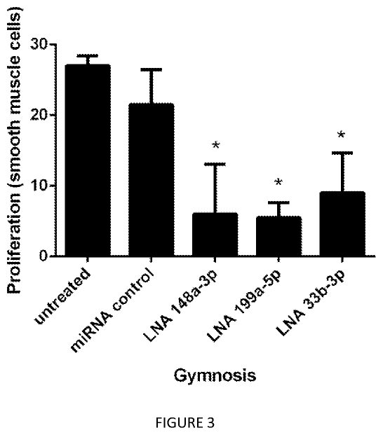

Using a gymnosis protocol, we verified a significant reduction in cell proliferation using the following miRNA-specific inhibitors: 148a-3p LNA, 199a-5p LNA and 33b-3p LNA. All three inhibitors strongly reduce the proliferation of smooth muscle cells (FIG. 3).

1. A method for treating uterine leiomyoma comprising administering to an individual in need there of a nucleic acid inhibitor of one or more miRNAs selected from the group consisting of miR-148a-3p, miR-199a-5p and miR-33b-3p.

2. The method of claim 1, wherein said inhibited miRNA is miR-148a-3p.

3. The method of claim 1, wherein said inhibitor is an oligonucleotide comprising at least 5 nucleotides capable of of binding to said one or more miRNAs by complementary base pairing.

4. The method of claim 3, wherein said oligonucleotide is able to pair at least 40% of its bases with the bases of said one or more miRNAs.

5. The method of claim 1, wherein said miRNA inhibitor comprises a locked-nucleic acid (LNA)-based oligonucleotide.

6. The method of claim 5, wherein said LNA-based oligonucleotide is at least partially complementary to said miRNA(s).

7. The method of claim 6, wherein said LNA-based oligonucleotide is at least about 40%, 50%, 60%, 70%, 80%, 90% or 100% complementary to said miRNA(s).

8. The method of claim 5, wherein said LNA-based oligonucleotide comprises an antisense oligonucleotide with perfect sequence complementary to the miRNA target.

9. The method of claim 5, wherein said LNA-based oligonucleotide comprises between 5 and 27 nucleotides and it comprises at least one locked nucleic acid.

10. The method of claim 1, wherein said inhibitor comprises an anti-miR.

11. The method of claim 10 wherein said anti-miR comprises between 5 and 27 nucleotides and it has at least about 40% of the bases complementary with the bases of said miRNA.

12. The inhibitor method of claim 1, wherein said inhibitor is administered without using a transfection agent.

13. The method of claim 1, wherein said inhibitor is administered to uterus cells by sonoporation.

14. The method of claim 1, wherein said inhibitor is administered using an intrauterine device medicated for release of said inhibitor.

15. The method of claim 1, wherein the at least one inhibitor of one or more miRNAs selected from the group consisting of miR-148a-3p, miR-199a-5p and miR-33b-3p is formulated as a pharmaceutical composition comprising at least one pharmaceutically acceptable vehicle and/or excipient.

16-17. (canceled)

18. An intrauterine device comprising or having contained therein at least one inhibitor of an miRNAs selected from the group consisting of miR-148a-3p, miR-199a-5p and miR-33b-3p, and the intrauterine device is medicated or fabricated for release of said inhibitor.
